(19) United States
(12) Patent Application Publication
(10) Pub. No.: US 2012/0003728 A1
(43) Pub. Date: Jan. 5, 2012

(54) Scalable Portable Sensory and Yield Expert System for BioMass Monitoring and Production

(75) Inventors: Mark Allen Lanoue, Long Beach, MS (US); Janek Kaliczak, Aptos, CA (US)

(73) Assignee: Mark Allen Lanoue, Long Beach, MS (US)

(21) Appl. No.: 12/828,362

(22) Filed: Jul. 1, 2010

(51) Int. Cl. C12M 1/34; G02F 1/01; F21V 33/00; A01G 31/02

(52) U.S. Cl. 435/288.7; 47/60; 359/238; 362/253

(57) ABSTRACT

The invention relates to the field of algae biofuel production, in particular to methods and means of physical action on biological structures of photosynthesing microorganisms, phototrophic algae in particular. The invention can be used for obtaining biofuel from algae, as well as for the pharmaceutical, cosmetic and foodstuff industries. In the process of the method implementation radiation of cultivated solution of photosynthesizing microorganisms/phototrophical algae is carried out by the action of electromagnetic waves of a selected intensity. Stimulation of increasing photosynthesizing microorganisms/phototrophical algae biomass is obtained by the interaction of electromagnetic wave and biological cell. Irradiation of cultivated solution of photosynthesizing microorganisms/phototrophical algae is performed by electromagnetic waves originating from specified sensor mechanisms mounted about the acrylic or plastic-based stackable tubular bioreactor. Nutrients, carbon dioxide and other dissolved substances are monitored by this sensor system which is controlled by a supercomputer-based control mechanism.

### BACKGROUND OF THE INVENTION

The present invention is in the technical field of biomass technology. More particularly, the present invention is in the technical field of a stackable environmentally controlled biomass production, yield enhancement and sensory system/architecture.

Conventional biomass production systems take up large areas and are not well suited for total unconditional environmental control. It is difficult to use the prior art in this field in small land space and densely populated areas where the current energy infrastructure is typically located and in areas where there simply is not enough land space to establish the prior art in biomass production. The major obstacle to systems growing biomass material with just sunlight is the growth period could be restricted by the latitude and diminished sunlight the growth system is acquiring and in sunlit areas the day night cycle reduces the potential growth time that could be available. The prior art also lacks the sensory and simulated plant cycle and weather protecting infrastructure for ultimate yield, and life cycle in changing climates. The current systems are used in large land spaces and use conventional nutrient systems and CO2 feeds. The difficulties of bringing such devices close to the current infrastructure and near power centers is that it is very difficult in the US and impossible in smaller densely populated northern or southern latitude countries; the current nutrient systems and CO2 feeds do not utilize advanced sensory systems at the nutrient tanks and the refinery tanks as well as throughout the entire system in order to produce the best yield of biomass product, while monitoring the closed system via the use of current infrastructure output of CO2 that can be used in the system, while off gassing O2 into the environment. Further, it is not an uncommon experience to realize that the current systems and architecture will not work in hostile environments such as extreme cold, etc. Further, the current devices and infrastructure do not remotely monitor the system with security feeds and prevent anti-internet attacks to the system. The proposed invention will produce a solution for a high tech, low land use, monitored, sensory, high yield biomass production system and an environment that will utilize current infrastructure and output from current (existing) power production facilities while being powered by environmentally controlled and environmentally (existing available) friendly sources.

### SUMMARY OF THE INVENTION

The present invention is a Biomass solution for a high tech, low land use, monitored, sensory, high yield biomass production system and environment that will utilize current infrastructure and output from current (existing) power production systems while being powered by environmentally controlled and existing, and available friendly sources.

The Spectral Biomass Growth Control and Monitoring System will monitor and control all aspects of the growth cycle and production of a biomass in an enclosed liquid medium. A plurality of growth monitoring sensors will send information on the growth and state of the biomass via a wireless mesh network to a master Expert System that can control the movement of biomass material, concentration and level of dissolved nutrients, growth specific gasses and state of the biomass, by sending signals back through the mesh network to the spectral growth monitor (Expert System) that can control relays, pumps, and lighting sources; therefore, promoting the growth of the biomass and facilitating the transport, extraction and production of the product (output) produced by the biomass.

The proposed invention consists of an environmentally controlled enclosure, growth enhancement monitoring, and a sensor-based stacked unit closed loop system for biomass production. The system will incorporate waste from the existing infrastructure (such as CO2 waste from coal energy production or other similar sources). Wavelength specific light panels that totally enclose the growth modules, electrical stimulation of biomass nutrients and sensor monitored regimes will be used to maximize yield of the biomass material. In addition, the system will make use of alternative non-petroleum sources to power the environmentally friendly system. The systems impact on the environment will be minimized and take the form of O2and innocuous compost material. The sensor units will be strategically placed in order to monitor nutrient and CO2 distribution, input and output levels and dark/light exposure cycles for maximum yield and growth cycle management. The entire system will be enclosed and based on a greenhouse model with stackable units in order to take advantage of small dense land spaces and the ability to place the systems close to existing infrastructure. The system will be a sealed, closed loop system that will have automated vented O2 emission systems and retention systems for O2 and CO2 storage. The sealed system would have environmental controls so that it could be used in harsh outdoor environments, including extremes such as hot, cold, dark, dry, and wet conditions or in totally enclosed underground environments The entire system would be based on manufactured land space stackable modules for easy maintenance and clean out, designed for cyclical biomass production and factory power structure. This system also lends itself to portability utilizing heavy equipment movers and standard shipping container environments. Such equipment movers could be heavy land movers or standard train based transport systems.

### BRIEF DESCRIPTION OF THE DRAWINGS

FIG. 1

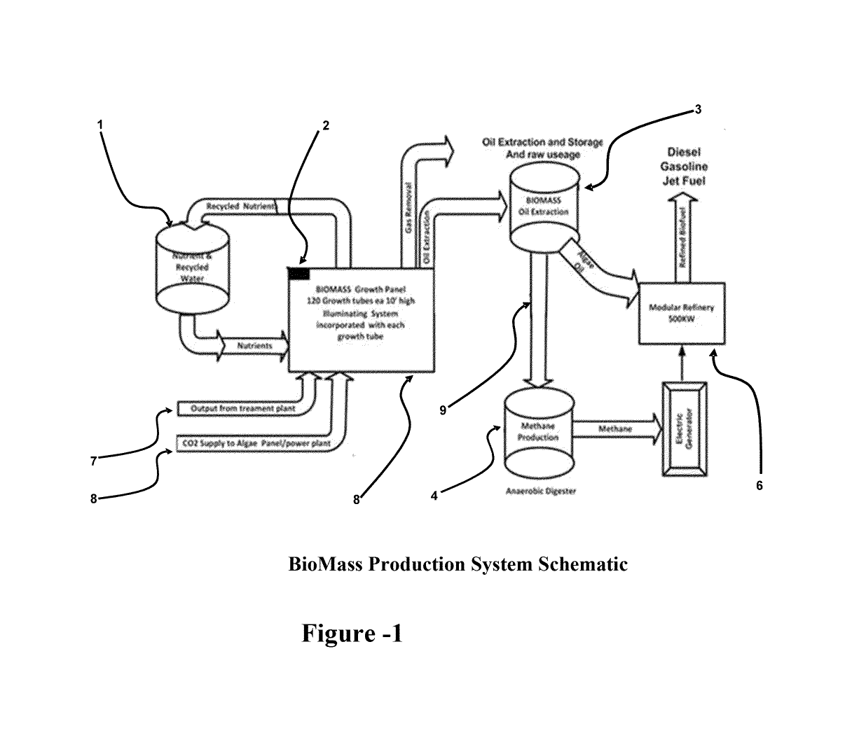

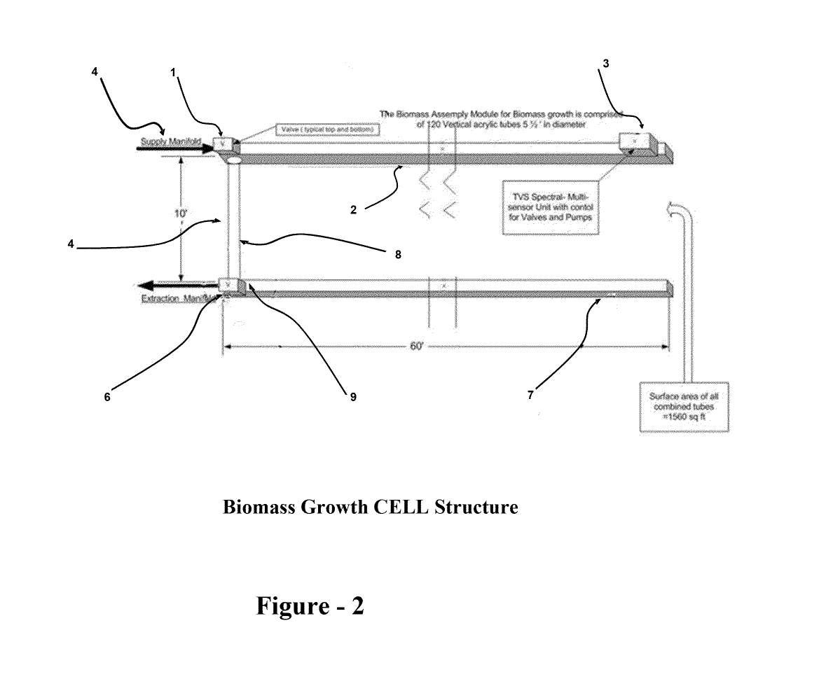

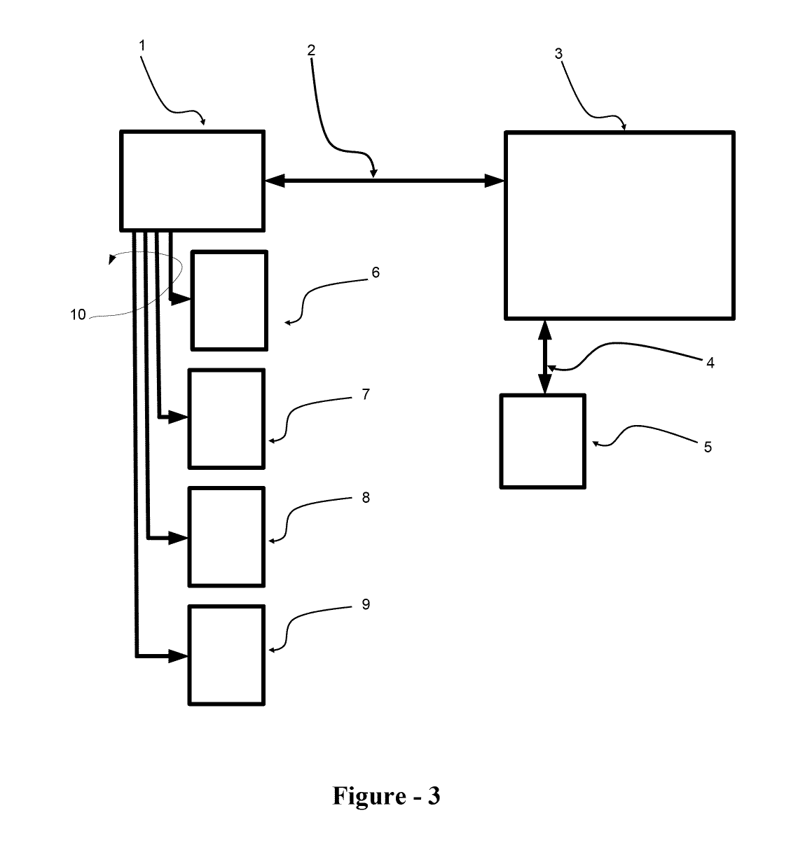

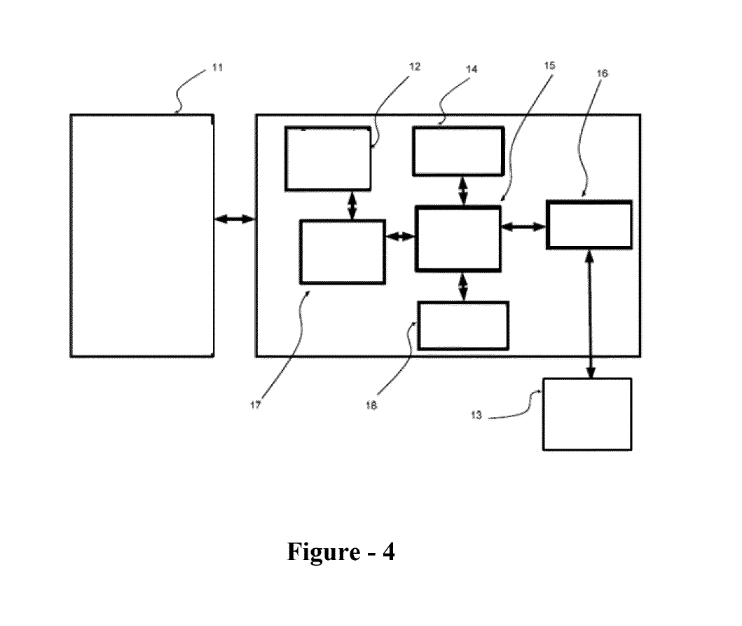

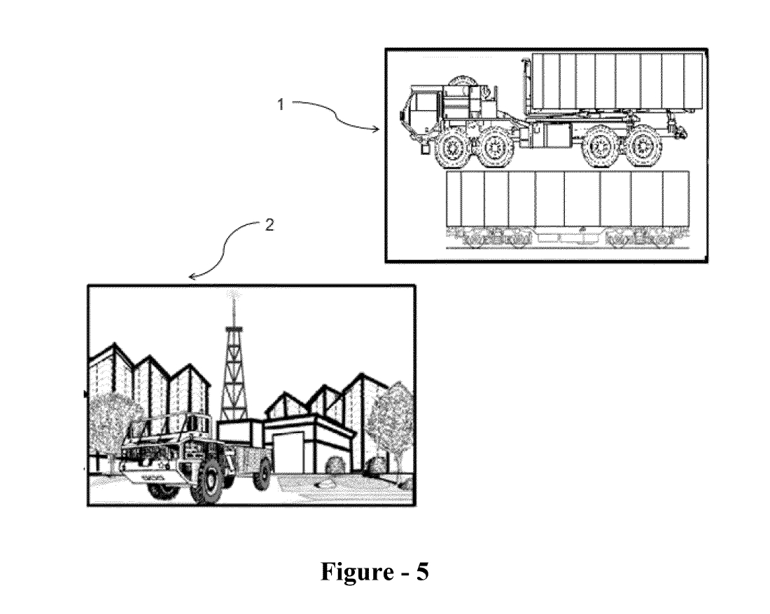

The biomass production system schematic FIG. 1, The entire system schematic shows the major system components. These components are
1. A storage source for Nutrients, recycled water for the biomass growth cell
2. A spectral monitoring sensor that monitors the components, and growth of the fuel algae and reports this to the expert system
3. Biomass Algae Oil Lipid extraction which separates the Algae Oil Lipid from other extraneous biomass materials. The system is also monitored by the spectral sensor which sends the information to the expert system
4. Anaerobic Digester system which takes the non Algae Biomass materials and produces methane to power the motor generator, that supplies the plurality of electrical power for the entire system.
5. A growth cell containing a plurality of plastic growth tubes; a plurality of these cells makeup the entire system. The growth cell is monitored by a spectral sensor which sends the information to an expert system.
6. A electro plasma refinery that takes a plurality of the Algae Oil Lipid and produces aviation fuel, diesel oil, gasoline and propane as the deliverable products from this system
7. A supply of nutrient matter from the output of a sewage treatment plant
8. A supply of CO2 from the output of an electrical generation power plant.
9. Biofuel Extraction residual materials to Methane bioreactor

FIG. 2

The Biomass Growth Cell FIG. 2, represent a plurality of growth cells.

FIG. 2 represents a typical biomass growth CELL panel. The cell could have plastic tubes arranged in either a vertical or horizontal configuration. Each of the plastic tubes is surrounded by a lighting blanket made with an OLED light panel wrapped as a blanket around the tube. The system also has a supply manifold for all materials and an extraction manifold for extraction of all materials. There are valves, pumps and cross connecting piping that represent a plurality of each biomass growth cell. Each cell has a spectral sensor that connects to the expert system.
1. A representation of a control valve in the growth cell
2. A representation of a SUPPLY MANIFOLD for the biomass growth CELL. Each biomass growth tube connects to the supply manifold opposite to the other manifold
3. A Spectral Sensor which monitors biomass material movement as pumped through the system, monitors nutrients, gases and algae fuel growth maturity through the cell and sends this information to the expert system
4. Growth tube represents a plurality of tubes in the growth cell. The tube will have an optimum internal diameter of 5½ inches to provide the optimum growth surface area for the algae fuel to receive spectrally tuned lighting from an organic light emitting diode (OLED) panel wrapped around a plurality of growth tubes in a typical growth cell.
5. A representation of an EXTRACTION MANIFOLD for the biomass growth CELL. Each biomass growth tube connects to the extraction manifold opposite to the other manifold
6. A quick release sliding mechanism for each growth tube in the biomass growth cell. This sliding coupling allows quick replacement and maintenance of a plurality of growth tubes in a biomass growth cell.
7. A representation of an Extraction MANIFOLD for the biomass growth CELL.
8. Biomass Growth cylinder, typical of a plurality of tubes, in the Biomas Growth CELL structure, representing many such Growth CELL's with a median diameter of 5½ inches, and a median height of 120 inches. Spectrally tuned organic light emitting diode (OLED) panel wrapped around biomass growth cylinder representing a plurality of such cylinders.
9. Biomass Growth tube connection to SUPPLY and EXTRACTION manifold is with slide on slide off gliders providing mechanical connection to both manifolds and gaskets on both ends to provide a tight seal for a plurality of all such connections in the system

FIG. 3

Referring now to the invention in more detail, in FIG. 3 Labeled from 1 thru 10 in further detail.

1). Spectral Growth Monitoring Sensor system. The growth monitor includes a number of photosensors arranged so that the device can monitor the growth of biomass materials in an enclosed environment. The monitoring window will have to be spectrally clear in order to allow the penetration and reflection of the specified spectral frequencies needed to monitor the state and growth process of the biomass. The multitude of photosensors in the Spectral Growth Monitoring system could have a removable component enabling replacement due to various levels of damage or to make a change in the spectral frequencies of the photosensor units. The Spectral Growth Monitoring system will communicate with the Master Expert system via a mesh network (2);

2) A mesh network system could enable routing of data, voice and instructions between the Spectral Growth Monitoring Nodes and the Expert System. The mesh network maintains uninterrupted connections and spontaneous reconfiguration around broken or blocked paths by “hopping” from node to node until the destination is reached, resulting in a very reliable network. Mesh networks differ from other networks in that the component parts can all connect to each other via multiple hops; generally they are not used in a mobile capacity. The mesh network will operate across multiple radio bands. For example, there is an option to communicate node to node on 5.2 GHz or 5.8 GHz, and node to client on 2.4 GHz (802.11). This action is accomplished using SDR (Software-Defined Radio). The network will also have a self testing feature that will constantly test the Spectral Growth and Control nodes and signal the Expert System if there are any deficiencies or irregularities in the network or if maintenance needs to be performed.

3) The Expert System

4) The mesh network interface to the remote control system carried by the system operator(s)

5) The systems operator's remote control system running on the mesh network to exercise override and control the BIOMASS system.

(6, 7, 8, 9) A control relay attached to the Spectral Growth Monitor to control valves, switches, pump motors, lighting controls, and safety systems; typical low voltage control modules.

10) Low Voltage control modules connected to Spectral Growth Monitor via local mesh network.

FIG. 4

Referring now to the invention in more detail, in FIG. 4 Labeled from 1 thru 10 in further detail.

11) Spectral Sensor Removable Sensor Module

12) PIXELARM Sensor control

13) Remote low voltage control linked to Spectral Sensor Growth Module via local mesh network.

14) External Control Module.

15) Main Spectral Imager Growth Module CPU

16) Local Mesh interface module

17) Spectral Imager Growth Control Module Calibration and control

18) Master Mesh Network Control Module to Master Expert System

FIG. 5

In further detail, referring to the invention in FIG. 5. Labeled from 1 thru 3 in further detail. Typical for the biomass production system, producing usable biomass material and packaged in multiple forms of transport and also of either single story production shelters, or multistory production centers.

1) The biomass self contained production system

2) The biomass self contained production system on portable, movable transport systems

3) The biomass self contained production system in either single story or multiple story production structures.

1. A system for optimizing plant growth in a liquid environment within an enclosed growth chamber, comprising: illumination means for controlling spatial, temporal and spectral characteristics of illumination surrounding the growth chamber, said illumination means comprising an array of individually actuable light sources, made up of organic light emitting diodes (OLED), is a light-emitting diode (LED) whose emissive electroluminescent layer is composed of a film of organic compounds that emit light when an electric current passes through it. This layer of organic semiconductor material is formed between two electrodes, where at least one of the electrodes is transparent. Just like passive-matrix LCD versus active-matrix LCD, OLEDs can be categorized into passive-matrix and active-matrix displays. Active-matrix OLEDs (AMOLED) require a thin-film transistor backplane to switch the individual pixel on or off, and can make higher resolution and larger size displays possible. The individual pixels then can be turned on as a group to produce light in the visible spectrum and containing frequencies that are particular to maximizing the group of the biomass material through photosynthesis. This array would surround the group tube containment vessel whether the tubes were vertical or horizontal configurations and supply lighting in the required frequencies needed to maximize plant growth and to minimize the power comsumption of said system; this would be in tandem with other lighting support such as centered mercury and other prior art lighting for agricultural based systems. The system can also supply specific on and off spectral frequencies, a continuous rolling mode switching the spectral frequencies into a dynamic moving configuration. This control allows for specific control over the circadian day night rhythm to increase plant growth. The lighting system is arranged as a blanket around the growth tubes within the system and having differing spectral wavelengths, and means for individual control and modulation of said light sources within each growth tube of said said system So that a series of growth tubes would create a cell of tubes either in a vertical or horizinal configuration. The biomass detection means comprising at least one imaging device which can identify and map location and quantity of plants in a growth medium within the growth cell configuration. The plant stress detection means for acquiring spatially distributed image data which characterize plant vigor and stress within the growth chamber, according to said growth tube cell patterns, said plant stress detection means comprising at least one imaging device selected from the group consisting of multispectral imagers, and hyperspectral imagers; environmental monitoring means for monitoring a plurality of environmental parameters that affect plant growth in a liquid within a growth chamber; environmental control means for controlling each of said environmental parameters; and an expert system coupled to receive data generated by said biomass detecting means, said plant stress detection means, said imager and said environmental monitoring means, and coupled to control said illumination means and said environmental control means; wherein said expert system contains a knowledge base that includes heuristic information, a plant database containing cultivation diagnostic and spectral information for plants growing within the growth chamber, and plant biomass and stress detection algorithms; and said expert system is trained to regulate said illumination, environmental parameters and the motors pumps and valves that control movement of plant material, nutrients and gases within the growth tube chambers as a plurality of the whole. This expert system is trained so as to achieve optimized uniform plant growth with minimized consumption of energy and materials, to diagnose deviations from optimal growth conditions, and to determine and implement remedial actions by adjustment of said illumination, dissolved gases and environmental parameters. Said expert control system would repeat indefinitely the expert control cycle to remove mature plants and materials from said environment to a predetermined process holding area and renew the expert process by bringing in startup plant growth materials and recycled and new nutrients and gases to repeat the process again until maturity was again retained and to repeat this process. The system contains growth modules which we have defined as a plurality of grow tubes in cell, and multiples of these cells in the system. Each growth cell contains a number of growth tubes arranged in either a vertical or a horizontal configuration for a plurality of all. Each cell with its growth module tubes has sensing control modules to monitor the biomass materials, gasses, nutrients, lighting, flow and maturity of the biomass. The biomass and the additional materials circulate within the plurality of cells until mature as defined by the expert control system. At maturity the biomass and all materials are taken to the extraction system which separates the algae fuel, also called algal fuel, algaeoleum or second-generation biofuel, is a biofuel which is derived from algae. The expert system which controls this entire process is connected through a wireless mesh network throughout the entire system which terminates at the Expert system which is trained to operate the entire production process

2. The system according to claim one separates the algae fuel organism into lipids, or oil and the remaining discarded materials, the algae's carbohydrate content can be fermented through anaerobic digestion into bioethanol and biobutanol or the plurality of the discarded materials can be placed into a bioreactor to produce methane for fueling the electrical generation system that supplies power to the entire system. The volumetric use of this discarded material to make one or the other of the manufactured materials in this process will be controlled by the expert system.

3. The system according to claim 1 and claim 2 will take the discarded materials after the separation of algae lipid fuel oil. Anaerobic digestion is a series of processes in which microorganisms break down biodegradable material in the absence of oxygen, used for industrial or domestic purposes to manage waste and/or to release energy. The result of this process is methane gas.

4. The system according to claim 1 will take the ALGAE OIL LIPIDS produced by the separation as described in claims 2 and 3 and use it directly in engines modified to use it directly without refining.

5. The system according to claim 1 will take the ALGAE OIL LIPIDS produced by the separation as described in claims 2 and 3 and use it as feedstock for the system oil refinery. There it can be transformed into fuel by hydro cracking (which breaks big molecules into smaller ones using hydrogen) or hydrogenation (which adds hydrogen to molecules). These methods can produce aviation fuel, gasoline, diesel, and propane. One type of algae, Botryococcus braunii produces a different type of oil, known as a triterpene, which is transformed into alkanes by a different process. The system is designed to take advantage of many different processes that are extremely small and portable in nature and would be controlled by the expert system through the Wireless mesh network (WMN).

6. The system according to claim 1 will take a plurality of sensors, spectral sensors, motor controllers, pump controllers, electro mechanical controllers and network these devices with the expert system. The Wireless mesh network (WMN) is a communications network made up of a plurality of radio nodes organized in a mesh topology that connects a plurality of the system growth cell modules . The system wireless mesh network consists of mesh clients, mesh routers and gateways. The mesh clients are often laptops, cell phones and other wireless devices while the mesh routers forward traffic to and from the gateways which may but need not connect to the expert system and internet. The coverage area of the radio nodes working as a single network is sometimes called a mesh cloud. Access to this mesh cloud is dependent on the radio nodes working in harmony with each other to create a radio network. A mesh network is reliable and offers redundancy for network communications in this network. When one node can no longer operate, the rest of the nodes can still communicate with each other, directly or through one or more intermediate nodes. Failures of components in this system will then be noticed quickly. FIG. 1 illustrates how the wireless mesh networks would be distributed to monitor plant growth, movement of nutrients, gases, control of valves, pumps in the system. The expert system can then be trained to self form and self heal by sounding an alarm when failures do occur for maintenance and replacement of failed devices. Wireless mesh networks can be implemented with various wireless technology including 802.11, 802.16, cellular technologies or combinations of more than one type. A wireless mesh network can be seen as a special type of wireless ad-hoc network. It is often assumed that all nodes in a wireless mesh network are immobile but this need not be so. The mesh routers may be highly mobile. Often the mesh routers are not limited in terms of resources compared to other nodes in the network and thus can be exploited to perform more resource intensive functions. In this way, the wireless mesh network(WMN) differs from an ad-hoc network since all of these nodes are often constrained by resources. The plurality of components of this fuel production system depend on the WMN to control and direct a plurality of time dependent functions and actions for full process under Expert System control.

7. The system according to claim 1 will take a plurality of the methane gas produced in the anaerobic digestion process and use this methane gas in electrical generation equipment to supply electrical power to a plurality of the system and devices requiring electrical power

8. The system according to claim 1, further comprising a growth zone monitoring imager for identifying and mapping the growth of plants within the liquid medium contained in the chamber, according to said expert grid cell pattern.

9. The system according to claim 1, further comprising a network communications link between said expert system and a remote terminal which includes a machine/human interface, whereby a remotely situated supervisory individual may communicate with and override said expert system and provide control functions outside of the expert system. This supervisory individual could also retrain the Expert System with new values.

10. The system according to claim 6, wherein said interface includes multiple display means for displaying data from said detection and monitoring means, and diagnostic and environmental control determinations from said expert system to multiple repeaters within the system, even including remote wireless machine/human interface.

11. The system according to claim 6, wherein said environmental control means comprises delivery and control systems for each of said environmental parameters.

12. The system according to claim 5, wherein said environmental parameters include at least one parameter selected from the group consisting of temperature, plant nutrients, carbon dioxide and other gases, water and nutrients and comparable data by which to compare to. And volumetric measurements to assess the movement of the biomass through said array cell structure

13. The system according to claim 6, wherein the expert system adjusts said delivery and control systems based on a comparison of data from said monitoring and detection systems with optimum conditions stored in the plant database, using a heuristic technique that can be modified by the machine/human interface.

14. The system according to claim 1, wherein said expert system controls operation of said illumination means in response to said spatially distributed image data from said plant stress detection means to achieve a spatial, spectral and temporal distribution of illumination within the growth chamber that optimizes uniform plant growth, maximizes yield, and minimizes power consumption would be compared to the plant life maturity profile stored in the expert system and may be modified through the machine/human interface.

15. The system according to claim 1, wherein: periods of light and dark within the chamber are specific and the periods are controlled by the expert system and may also be modified through the machine/human interface.

16. The system according to claim 8, wherein said expert system controls said illumination and spectral frequency such that illumination is distributed to cells that contain biomass.

17. The system according to claim 10, wherein said expert system controls illumination on a cell by cell basis within said grid cell pattern, such that illumination is concentrated on growth tubes clustered into a cells within which plant stress is detected; also parameters of maturation.

18. The system according to claim 14, 15, and 16, wherein said light sources comprise an array blanket surrounding a plurality of growth tubes in a collection called a cell, each lighting blanket of organic light emitting diodes, which emit light at differing wavelengths on each pixel in the (OLED) array blanket, and which are distributed within each cell of the grid cell pattern according to a predetermined distribution.

19. The system according to claim 14, 15, 16, 17 wherein, when light is being distributed to biomass within a particular cell, the light energy is modulated according to a predetermined temporal pattern controlled by the Expert System.

20. The system according to claim 14, 15, 16, 17, wherein said predetermined temporal pattern includes modulating said light energy between first and second intensity levels at a predetermined frequency.

21. The system according to claim 14, 15, 16, 17 wherein said first intensity is zero and said second intensity has a fixed predetermined value.

22. The system according to claim 14, wherein said predetermined frequency is selected from a range between 200 and 1100 nm and through the machine/human interface as well as upgraded with further ranges as needed in the future such as Thermal and Middle (IR) wave ranges up to and including a spectral frequency of up to 2500 nanometers

23. A system for achieving optimized plant growth, comprising: a liquid growth chamber consisting of cells which contain a number of growth tubes either in horizontal or vertical configuration which is sealed off from an ambient environment; imaging means for acquiring and monitoring spatially spectral distributed plant growth information within said growth chamber, said imaging means comprising at least one device selected from the group of many cells consisting of a multispectral imager, and a hyperspectral imager; environmental monitoring means for acquiring and monitoring data regarding environmental conditions within said liquid growth chamber; illumination and environmental control means for controlling illumination and environmental conditions in said growth chamber; and an expert system that is coupled to said imaging means and said environmental means, said expert system being trained to analyze and evaluate crop growth conditions within the liquid growth chamber using a heuristic method, and to control said illumination and environmental control means to achieve optimized biomass crop growth and minimum consumption of energy and nutrients.

24. The system according to claim 6, further comprising a communications link between said expert system and a remote terminal which includes a machine/human interface, whereby a remotely situated individual may communicate with and override said expert system.

25. The system according to claim 6, wherein said interface includes display means for displaying data from said detection and monitoring means, and diagnostic and environmental control determinations from said expert system.

26. The system according to claim 17, wherein said expert system controls said illumination in response to said spatially distributed crop growth information to achieve a spatial, spectral and temporal distribution of illumination within the growth chamber that optimizes plant growth, maximizes yield and minimizes power consumption.

27. The system according to claim 20, wherein, when light is being distributed to biomass within a particular cell, and the light is modulated according to a predetermined temporal pattern.

28. The system according to claim 21, wherein said predetermined pattern includes modulating said light energy at a predetermined frequency.

29. The system according to claim 22, the intensity has a fixed predetermined value.

30. The system according to claim 23, wherein said predetermined frequency is selected from a range between 200 and 1100 nm with future enhancements up to 2500 na[n]ometers.

31. A system for controlling crop growth within a growth area, said system comprising: imaging means for monitoring spatially distributed crop growth information according to a predetermined grid cell pattern within said growth area; and illumination means for controlling spatial, temporal and spectral distribution of illumination within individual cells of said grid cell pattern in response to said crop growth and chemical information.

32. The system according to claim 23, wherein: said illumination means comprises a matrix of individually operable light sources that corresponds to said grid cell pattern; and said light sources include sources that emit light at a plurality of preselected wavelengths.

33. The system according to claim 26, wherein said illumination means comprises an expert system that receives crop growth and chemical information from said imaging means, and controls conditions within each cell of said grid cell pattern in response to said crop growth information, and expert system. Expert system has to do with sensory open and closed loop secure systems, monitoring, sensory systems, decision making, and database operation.

34. The system according to claim 27, wherein said expert system controls said illumination on a cell by cell basis, such that illumination is distributed only to those cells that contain biomass.

35. The system according to claim 28, wherein said expert system controls said illumination such that illumination is concentrated on cells within which plant stress and delayed maturation is detected.

36. The system according to claim 28, wherein said light sources comprise an array of organic light emitting diodes, which emit light at pre determined wavelengths, and which are distributed within each cell of the grid cell pattern according to a predetermined distribution.

37. The system according to claim 28, wherein, when light is being distributed to biomass within a particular cell, the light frequency is set according to a predetermined temporal pattern.

38. The system according to claim 31, wherein said predetermined pattern includes modulating said light energy at a predetermined frequency or modulating the frequencies separately or in concert with a predetermined frequency.

39. The system according to claim 32, wherein said first intensity is zero and said second intensity has a fixed predetermined value.

40. The system according to claim 32, wherein said predetermined frequency is selected from a range nominally between 200 and 1100 nm.

41. The system according to claim 1, wherein data from said biomass detection means is used for empirical biomass estimation.

42. The system according to claim 1 with all system components will operate in multimode configurations for all transport systems and all fixed based systems, single story, multistory, above or below ground.
